(19) United States
(12) Patent Application Publication
(10) Pub. No.: US 2013/0280343 A1
(43) Pub. Date: Oct. 24, 2013

(54) Food Products Derived From Cannabinoid-Administered Livestock

(71) Applicant: Justin Lee Roettger, Somerset, WI (US)

(72) Inventor: Justin Lee Roettger, Somerset, WI (US)

(21) Appl. No.: 13/452,205

(22) Filed: Apr. 20, 2012

(51) Int. Cl. A61K 35/20; A61K 31/35; A61K 35/54; A61K 35/12; A61K 35/34

(52) U.S. Cl. 424/535; 424/520; 424/548; 424/581; 514/454

(57) ABSTRACT

A process, wherein food-producing livestock are administered cannabinoid receptor agonists, is used to induce physiological and/or behavioral effects in the livestock. Food products are then derived from the livestock.

### TECHNICAL FIELD

The present invention relates to the technical fields of food, agriculture, and pharmacology, and specifically involves the administration of pharmacologically-active compounds to food producing livestock.

### BACKGROUND

Kobe beef is a high quality meat that is produced only in the Hyōgo Prefecture of Japan. It is named after the capital city of the Hyōgo Prefecture, Kobe. The beef, which is derived exclusively from the Tajima breed of Wagyu cattle, is considered to be a delicacy, and the agricultural techniques used to produce it are often viewed as a mystical folk art. Outside of Japan, Kobe beefs notoriety comes from tales of pampered cows that are hand-fed, drink beer, and receive massages. While not all Kobe cattle enjoy such spa-like luxuries, some do, and it is because of their lavish treatment that they yield some of the best quality meat in the world. Unfortunately, the high cost of providing cows with beer and massages precludes all but the very luckiest cattle from such opulent accommodation.

The beer given to Kobe cattle is provided as an appetite stimulant, especially during the summer months when the heat normally causes their hunger to wane. This ensures consistent weight gain and the desired accumulation of intramuscular fat, or marbling. Massages are given to the cows to relax their muscles, improving the distribution and softness of their sub-cutaneous fat, and increasing meat tenderness. Finally, the cattle are hand-fed in small, indoor enclosures, eliminating the need for foraging, and consequently reducing muscular development and further enhancing meat tenderness.

All of these effects—appetite stimulation, lowering residual muscle tension, and reducing physical activity—can also be actuated by the endocannabinoid system. The endocannabinoid system is a lipid regulatory system that is present in the brains and bodies of all vertebrates. Though it is perhaps the least-widely known biological system, the endocannabinoid system is involved in a large number of everyday physiological and psychological processes, including appetite control, nociception, mood regulation, and the formation and retention of memories.

Cellular receptors, called cannabinoid receptors, form the foundation of the endocannabinoid system. Only two cannabinoid receptors have thus far been identified, CB₁ and CB₂, though research has suggested the existence of at least one other. They are found primarily in the cells of the brain and nervous system, but are also present in the tissues of many other biological systems, including the digestive system, immune system, endocrine system, reproductive system, and circulatory system. When ligands bind with cannabinoid receptors, various biological responses are triggered. These ligands are ordinarily endogenously produced, but exogenous ligands are also capable of triggering responses regulated by the endocannabinoid system.

Cannabinoids are the name for the group of ligands that are capable of binding with cannabinoid receptors. There are three types of cannabinoids: endocannabinoids, phytocanabinoids, and synthetic cannabinoids. Endocannabinoids are naturally and endogenously synthesized in the bodies of humans and other animals. Phytocannabinoids are only produced in significant quantities by plants of the Cannabis genus; that is, Cannabis indica, Cannabis sativa, and Cannabis ruderalis. Note that not all cultivars of Cannabis contain high concentrations of cannabinoids; Cannabis cultivated for fiber and seed, commonly known as industrial hemp, contains levels far too low to be useful for eliciting any pharmacological effects. Finally, synthetic cannabinoids refers to those cannabinoids which are manmade.

The first endocannabinoid extracted from animal tissue, N-arachidonoylethanolamine (anandamide or AEA), was isolated from a porcine brain in 1992. At least 4 other endocannabinoids have subsequently been identified: 2-arachidonoyl glycerol (2-AG), 2-arachidonyl glyceryl ether (noladin ether), N-arachidonoyl-dopamine (NADA), and virodhamine (OAE). Produced endogenously by all vertebrates, these compounds are the usual ligands which trigger endocannabinoid system responses.

At least 85 phytocannabinoids have been isolated from the Cannabis plant. The three most abundantly produced are delta-9-tetrahydrocannabinol (Δ⁹-THC), cannabidiol (CBD), and cannabinol (CBN). Δ⁹-THC is of particular note, as it is the only major active constituent of the Cannabis plant, and is responsible for the physiological and psychological effects of recreational marijuana use. While a variety of other pharmacologically-active phytocannabinoids can be found in Cannabis, they are expressed in such low quantities that their presence ordinarily has no bearing on the effects experienced by subjects of Cannabis administration. CBD, and to a lesser extent, CBN, may be the only exceptions. The other phytocannabinoids include cannabigerol (CBG), cannabicheromene (CBC), cannabicyclol (CBL), cannabivarin (CBV), tetrahydrocannabivarin (Δ⁹-THCV), cannabidivarin (CBDV), cannabichromevarin (CBCV), cannabigerovarin (CBGV), and cannabigerol monoethyl ether (CBGM). The vast majority of all cannabinoids produced by the Cannabis plant are concentrated in the flower, particularly in the calyx and trichomes.

It is difficult to determine the total number of synthetic cannabinoids that have been created. A large number of them are analogs of endocannabinoids and phytocannabinoids. Certain newer compounds, however, show little structural relation to any naturally occurring compounds. Some notable synthetic cannabinoids include dronabinol, nabilone, rimonabant, JWH-018, dimethylheptylpyran, HU-210, and WIN 55,212-2.

Because the endocannabinoid system operates similarly in all vertebrates, and because it is primarily responsible for only a handful of processes, the effects of administering cannabinoids are very predictable. Cannabinoids mainly influence appetite, nociception, memory, mood, and energy. The way in which a cannabinoid impacts these different processes varies based on the particular cannabinoid administered, the method of administration, the dosage, and whether any counteracting substances were also administered.

Cannabinoids produce different effects based on whether they are receptor agonists, antagonists, or inverse agonists. Cannabinoid receptor agonists, such as the commonly-known Δ⁹-THC, initiate particular cellular responses upon binding with cannabinoid receptors. In vivo, these responses can lead to the physiological and psychological effects commonly associated with “getting high,” such as appetite stimulation, antinociception, short-term memory impairment, a sense of well-being, inhibition of aggressive behavior, muscle relaxation, and lethargy. On the other hand, cannabinoid receptor antagonists and inverse agonists, like rimonabant, can have opposite effects, such as appetite suppression, increased pain sensation, short-term memory improvement, depression, hostile or aggressive behavior, muscle tenseness, and increased energy and physical exertion.

Cannabinoids generally have extremely low toxicity. For example, the median lethal doses (LD₅₀s) of orally administered Δ⁹-THC, CBD, and CBN in mice are 21,600 mg/kg, 13,500 mg/kg, and 12,700 mg/kg, respectively. Compare this to the LD₅₀s for orally administered sodium chloride (table salt), ascorbic acid (vitamin C), and caffeine in mice, which are 4,000 mg/kg, 3,400 mg/kg, and 130 mg/kg, respectively. Notably, several attempts to establish the LD₅₀ of Cannabis plant matter in larger animals, including monkeys and dogs, have proved unsuccessful due to the limitations imposed by the animals' finite stomachs. The researchers conducting the studies found it physically impossible to feed the animals the raw quantities of Cannabis necessary to cause cannabinoid-induced death.

Contrasting their extremely low toxicity, cannabinoids generally have very low effective doses. For example, the median effective doses (ED₅₀s) for antinociception of orally administered Δ⁹-THC and CBN in mice injected with 5-Hydroxytryptamine (a nociceptive agent) are 1.0 mg/kg and 46.2 mg/kg, respectively. In humans, the lowest observed effect level (LOEL) for psychoactive effects induced by orally administered Δ⁹-THC is 0.04 mg/kg, or 3.2 mg for the average 80 kg adult human. As a result, the therapeutic indices (LD₅₀/ED₅₀) of most cannabinoids are astoundingly high. It would be extremely difficult, if not impossible, for a human or animal to suffer permanent damage due to an intake of cannabinoids, absent a targeted effort to cause such damage.

Because cannabinoids can be effective at doses so low relative to their lethal levels, it is easy to experiment and determine the appropriate dosage level for obtaining a particular pharmacological effect using a given cannabinoid, method of administration, and species of animal. Even significant overdoses, while capable of producing unpleasant temporary effects, are not likely to cause permanent injury.

Livestock have long been administered cannabinoids, incidentally, through Cannabis animal feed, as the non-cannabinoid nutritional benefits of Cannabis have been known for millennia. Hempseed, however, which is the structure of the Cannabis plant used as animal feed, contains no Δ⁹-THC. Furthermore, the Cannabis grown for animal feed is industrial hemp, and contains only trace quantities of Δ⁹-THC even in its calyx and trichomes; only recreational and medical Cannabis contain pharmacologically-significant amounts of cannabinoids. Also, industrial hemp contains a high quantity of CBD, a high-potency antagonist of cannabinoid receptor agonists. The high ratio of CBD:Δ⁹-THC present in industrial hemp, which often exceeds 1:1, prevents the already small quantities of Δ⁹-THC and other cannabinoid receptor agonists from eliciting any pharmacological effects with positive efficacy. For example, the Cannabis that farmers cultivate for subsidy in the European Union, and are legally permitted to grow in Canada, is subject to a Δ⁹-THC maximum of 0.3% with a CBD:Δ⁹-THC ratio requirement of ≧2:1. Proposed federal legislation here in the U.S. would adopt the same 0.3% dry weight limit on Δ⁹-THC content for industrial hemp production. Recreational marijuana, on the other hand, often has a Δ⁹-THC content of >10% and a Δ⁹-THC:CBD ratio of 5:1-10:1.

### SUMMARY

In present invention, one or more kinds of cannabinoid receptor agonists are administered to livestock from which food products are derived. These cannabinoids may be phytocannabinoids, endocannbinoids, or synthetic cannabinoids, or any other chemical compounds or the prodrugs of any chemical compounds which binds to cannabinoid receptors and exhibit a positive efficacy. By interacting with the livestock's endocannabinoid systems, the cannabinoids generate significant physiological and/or behavioral effects in the animals. Some of these effects may be desirable to the producers and consumers of the food products derived from the livestock.

Administration is the process of directing, advancing, or implementing an event or set of circumstances, for the purpose of eliciting one or more pharmacological effects, which results in the introduction of cannabinoids into the bodies of livestock. Any route of administration may be used, including but not limited to oral, intravenous, or inhalational administration. Administrating the cannabinoids may be done actively, e.g. by feeding hogs compressed pellets containing dried Cannabis plant matter, or passively, e.g. when deer graze on Cannabis plants that have been cultivated for their consumption.

A sufficient degree of cannabinoid receptor agonists must be introduced into an animal's body to actually elicit a significant pharmacological effect with positive efficacy. This may be accomplished over time with multi-dose or chronic administration, or immediately with the administration of a single acute dose. An effect is significant if it is of such a type and degree that its origin can positively be attributed to the administration of cannabinoids, and is reasonably apparent to some individual during the ordinary course of raising livestock, deriving food products from that livestock, or consuming the food products produced by that livestock. Introducing small quantities of cannabinoids into the bodies of livestock which elicit no ordinarily observable effects, either in the animals or in the food products they produce, is insufficient to establish the existence or occurrence of a significant pharmacological effect, as is using unordinary investigatory methods to identify effects which would remain undetected otherwise.

When sufficiently large levels of cannabinoid receptor agonists are administered to livestock, one significant pharmacological effect that may be induced is the accumulation of a non-trivial quantity of cannabinoids in the livestock's bodies, such as in the adipose tissue, milk, or egg yolks. In these instances, some food products derived from such livestock may contain pharmacologically significant quantities of cannabinoids as a result of the cannabinoid receptor agonists previously administered. The quantity of cannabinoids in a derived food product is pharmacologically significant if the cannabinoids present in a USDA serving size of the food product, or another article of food prepared or processed using the food product, meet or exceed the LOEL for eliciting psychoactive effects in the average adult human being with an acute oral dose, as would occur when the food product is consumed.

A common definition of the term livestock refers only to mammals which have been domesticated for an agricultural purpose. Here, however, livestock is meant in a broad sense to refer to all animals that have endocannabinoid systems, are capable of being administered cannabinoids, and are edible or produce products which are edible. This definition includes cattle (Bos primigenius taurus), sheep (Ovis aries), goats (Capra aegagrus hircus), swine (Sus scrofa domestica), deer (e.g. Odocoileus virginianus), poultry (e.g. Gallus gallus domesticus), fish (e.g. Salmo salar), and potentially any other vertebrate for which cannabinoids can be administered and food can be derived.

Finally, food products are any products or commodities produced by the bodies of livestock that are fit and intended for oral consumption. This includes meat, milk, eggs, fat, organs, and any other edible substance or thing which may be harvested, collected, or obtained—i.e., derived—from livestock with the purpose of using it as food. Products which require cooking or some other form of processing prior to consumption, and products which are intended for animal rather than human consumption, are not excluded from this definition. Those tissues, organs, or other animal products which are derived for purposes other than oral consumption, however, are not food products.

### BRIEF DESCRIPTION OF DRAWINGS

FIG. 1 depicts a T-bone steak derived from cattle that had been fed a diet including Cannabis.

### DETAILED DESCRIPTION OF ILLUSTRATIVE EMBODIMENTS

Reference will now be made to several potential embodiments of the present invention. Each detail is provided as an explanation of the present subject matter, and should not be construed to narrow the metes and bounds of the invention's broader conception. While the subject matter will be described in conjunction with particular embodiments, it will be apparent to persons having ordinary skill in the art that many further modifications and variations can be made to the present invention without departing from the spirit or scope thereof. Thus, it is intended that the present subject matter covers such modifications and variations, inasmuch as they would come within the scope of the appended claims.

In one embodiment of the present invention, Tajima beef cattle are finished on a diet consisting of 80% temper rolled barley, 10% haylage, and 10% dried Cannabis sativa plant matter of various cultivars. The Cannabis sativa used has an approximate Δ⁹-THC content of 8.0%, CBN content of 4.0%, CBD content of 0.5%, and a Δ⁹-THC:CBD ratio of 16:1. After consuming the Cannabis for several days, the cattle experience physiological and behavioral effects as a result of the cannabinoids present in the ingested plants. Some of these potential effects may be desirable to cattle farmers, such as the pacification of aggressive cattle, an increase in beef quality, and an increase in carcass yield due to appetite stimulation and weight gain.

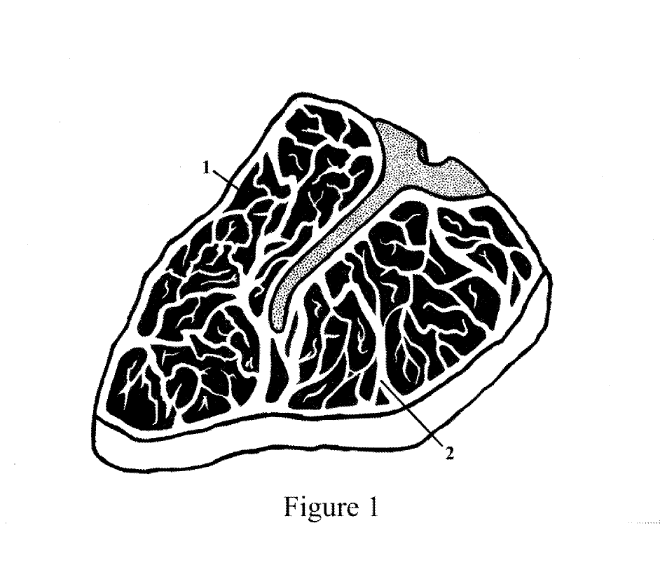

FIG. 1 depicts a T-bone steak obtained from cattle which had experienced these effects. It exhibits significantly improved characteristics due to the cannabinoids that were present in the ingested fodder. The lethargy induced by the cannabinoids, or the decrease in the livestock's energy and vigor, corresponded with a reduction in the livestock's net daily movement, and consequently reduced the cattle's muscular development. In combination with the cannabinoids' muscle relaxant and appetite stimulation effects, this resulted in superior meat tenderness 1 and enhanced marbling 2.

In a second embodiment of the present invention, dairy water buffalo are grazed in a pasture cultivated for their consumption which contains cereals, legumes, and Cannabis indica “Himalaya Gold” (a hardy, outdoor cultivar of Cannabis). The approximate Δ⁹-THC, CBN, and CBD levels of the Himalaya Gold Cannabis cultivar are 10.0%, 1.0%, and 0.3%, respectively. The animals are left to graze according to their preference, and all three types of plants are consumed in significant quantity. Particularly, the water buffalo consume about 32 kg of raw plant material per day, of which an average of 4 kg is Cannabis. Thus, roughly 400 g of Δ⁹-THC is orally administered to the animals each day.

The cannabinoids present in the Himalaya Gold Cannabis induce physiological effects which affect the composition of the water buffalo's milk. More specifically, several fat-soluble cannabinoids, including Δ⁹-THC and 11-Hydroxy-Δ⁹-tetrahydrocannabinol (11-OH-Δ⁹-THC; an active metabolite of Δ⁹-THC), accumulate in the milk during lactogenesis. The cannabinoid-containing raw milk is then harvested from the water buffalo by use of a rotary parlor milking station. The milk, of which each water buffalo produces an approximate 20 L per day, accumulates Δ⁹-THC at an average transfer rate of 0.15%, and thus contains around 30 mg of Δ⁹-THC per L. The USDA serving size of one cup, therefore, contains approximately 7 mg of Δ9-THC, which is an effective dose for inducing moderate psychoactive effects in adult human beings.

In a third embodiment of the present invention, finishing hogs are supplied with water which has been fortified with an alcohol extract containing Δ⁹-THC, CBN, and many other phytocannabinoids. The extract is a tincture formed by steeping six-months-dried Cannabis indica “Himalaya Gold” in a solution of 70% ethanol and 40% water at a ratio of 1 g of Cannabis per 5 mL of solution for two weeks, followed by straining. Assuming total dissolution, the solution contains 20 mg of Δ⁹-THC per 1 mL of tincture. The extract is then added to the hogs' water supply at a ratio of 10 mL of extract per 32 L of water. The average finishing pig, which drinks about 16 L of water per day, thus orally consumes approximately 5 mL of extract including 100 mg of Δ⁹-THC each day.

Similar to the previous embodiment, where the cannabinoids accumulated in the water buffalo's milk, a significant quantity of the Δ⁹-THC and other cannabinoids administrated to the hogs, as well as a portion of their active metabolites (which are also cannabinoid receptor agonists, e.g. 11-OH-Δ⁹-THC), accumulate in the hogs' adipose tissue. When the hogs are later harvested for meat, the resulting pork includes these cannabinoids stored in the fat. At an average Δ⁹-THC storage rate of 0.1% in adipose tissue, bacon slices obtained from these hogs, each containing 2 g of fat, would contain 2 mg of Δ⁹-THC a piece. If this bacon is cooked at a relatively low temperature and in a liquid base, e.g. in creamed spinach soup at 145° F., no Δ⁹-THC would be lost to thermal degradation or discarded in leftover grease. A creamed spinach soup cooked at 145° F. that contains three slices of bacon per bowl, therefore, would yield a Δ⁹-THC content close to 6 mg per serving, and again be effective at eliciting moderate psychoactive effects in adult human beings of average size.

In a final embodiment of the present invention, soft gel capsules containing 1 μg of dimethylheptylpyran dissolved in cod liver oil are twice-daily orally administered to free range broiler chickens. Dimethylheptylpyran is an extremely potent cannabinoid receptor agonist. It significantly calms the chickens, making it much easier to handle them and preventing them from becoming excessively boisterous. In addition, the dimethylheptylpyran inhibits aggressive behavior, eliminating instances of cannibalism and consequently preventing the need for debeaking, a practice which many consider to be cruel.

What is claimed is:

1. A process for improving livestock characteristics, comprising:
administering cannabinoid receptor agonists to livestock;
the cannabinoid receptor agonists producing one or more significant pharmacological effects with positive efficacy in the livestock; and
deriving one or more food products from the livestock.

2. The process of claim 1, further comprising the administration being oral.

3. The process of claim 1, further comprising the administration being chronic.

4. The process of claim 1, further comprising the cannabinoid receptor agonists being synthetic cannabinoids.

5. The process of claim 1, further comprising the cannabinoid receptor agonists being Δ⁹-THC.

6. The process of claim 1, further comprising a significant pharmacological effect produced by the administration of cannabinoid receptor agonists being appetite stimulation.

7. The process of claim 1, further comprising a significant pharmacological effect produced by the administration of cannabinoid receptor agonists being lethargy.

8. The process of claim 1, further comprising a significant pharmacological effect produced by the administration of cannabinoid receptor agonists being a reduction in residual muscle tension.

9. The process of claim 1, further comprising:
the food products derived from the livestock being meat; and
a significant pharmacological effect produced by the administration of cannabinoid receptor agonists being a change in the firmness, texture, and/or marbling of the meat.

10. The process of claim 1, further comprising:
a significant pharmacological effect produced by the administration of cannabinoid receptor agonists being the accumulation of one or more cannabinoids within the bodies of the livestock; and
the derived food products containing a pharmacologically significant quantity of cannabinoids.

11. A food product obtained by the process of claim 1.

12. A food product obtained by the process of claim 5.

13. A food product obtained by the process of claim 9.

14. A food product obtained by the process of claim 10.

15. The food product of claim 11, further comprising being meat.

16. The food product of claim 11, further comprising being milk.

17. The food product of claim 11, further comprising being eggs or meat from poultry.

18. The food product of claim 15, further comprising the derived meat containing a pharmacologically significant quantity of cannabinoids.

19. The food product of claim 16, further comprising the derived milk containing a pharmacologically significant quantity of cannabinoids.

20. The food product of claim 17, further comprising the derived eggs or meat containing a pharmacologically significant quantity of cannabinoids.
